(19) United States
(12) Patent Application Publication
(10) Pub. No.: US 2016/0213560 A1
(43) Pub. Date: Jul. 28, 2016

(54) AUTOMATED CARDIOPULMONARY RESUSCITATION (CPR) DEVICE

(71) Applicant: Renata Sturdivant, Pflugerville, TX (US)

(72) Inventor: Renata Sturdivant, Pflugerville, TX (US)

(21) Appl. No.: 15/002,278

(22) Filed: Jan. 20, 2016

Related U.S. Application Data
(60) Provisional application No. 62/105,639, filed on Jan. 20, 2015.

(51) Int. Cl. A61H 31/00; A61N 1/39

(52) U.S. Cl. CPC A61H 31/006; A61N 1/3987

(57) ABSTRACT

Systems and methods are disclosed for applying cardiopulmonary resuscitation (CPR) via an automated CPR device. The automated CPR device may configured to be secured to the chest of an individual, obtain data about the individual, and calculate compression parameters for applying chest compressions based on the obtained data. The automated CPR device may include one or more sensors for obtaining the data, and may also receive data via a user input system. The automated CPR device may include a defibrillator, and may be configured to monitor for a shockable heart rhythm, and administer an electric shock when a shockable rhythm is detected.

### CROSS-REFERENCE TO RELATED APPLICATIONS

The present application is a non-provisional application of and claims priority to pending U.S. provisional patent applications Ser. No. 62/105,639, filed Jan. 20, 2015, entitled “Automated Cardiopulmonary Resuscitation (CPR) Device,” the contents of which are hereby incorporated by reference in their entirety.

### BACKGROUND

This disclosure is related to devices for performing emergency procedures on an individual.

### SUMMARY

In certain embodiments, an apparatus may comprise an automated chest compression device configured to be secured to a chest of an individual. The automated chest compression device may include a compression cushion configured to be placed against the chest, and a motor to drive the compression cushion into the chest. The automated CPR device may also include a processor configured to calculate one or more chest compression parameters based on data about the individual, and control the motor to perform chest compressions based on the chest compression parameters.

In certain embodiments, an apparatus may comprise an automated cardiopulmonary resuscitation (CPR) device configured to be secured to a chest of an individual. The automated CPR device may include a compression mechanism configured to perform chest compressions on the individual, a defibrillator configured to deliver electrical shocks to the individual, and a processor configured to control the compression mechanism and the defibrillator. The processor may be configured to calculate one or more chest compression parameters based on data about the individual, and control the compression mechanism to perform chest compressions based on the chest compression parameters. The processor may further be configured to monitor the individual for a shockable heart rhythm, and control the defibrillator to deliver an electric shock when a shockable heart rhythm is detected.

In certain embodiments, a method may comprise attaching an automated cardiopulmonary resuscitation (CPR) device to a chest of an individual, detecting biometric measurements from the individual via sensors of the automated CPR device, calculating one or more chest compression parameters based on the biometric measurements via a processor of the automated CPR device, and applying chest compressions to the individual via a chest compression mechanism of the automated CPR device based on the chest compression parameters.

### BRIEF DESCRIPTION OF THE DRAWINGS

FIGS. 1A and 1B are an illustrative embodiment of an automated cardiopulmonary resuscitation (CPR) device;

FIG. 2 is another illustrative embodiment of an automated CPR device;

FIG. 3 is a flowchart of an illustrative method for an automated CPR device;

FIG. 4 is a flowchart of another illustrative method for an automated CPR device; and

FIG. 5 is an illustrative embodiment of an automated CPR device.

### DETAILED DESCRIPTION

In the following detailed description of the embodiments, reference is made to the accompanying drawings which form a part hereof, and in which are shown by way of illustration of specific embodiments. It is to be understood that other embodiments may be utilized and structural changes may be made without departing from the scope of the present disclosure.

Cardiopulmonary resuscitation (CPR) can be vital to maintaining blood circulation and oxygen to the brain in persons experiencing cardiac arrest. CPR may involve the application of repeated chest compressions to a subject. For example, according to International Liaison Committee on Resuscitation guidelines, chest compressions should be delivered at a rate of 100 per minute, with a chest compression depth of approximately 5 cm (2 inches).

During resuscitation efforts, pressure may be applied to the sternum in order to artificially compress the heart, which allows continued blood flow to vital organs in the event of cardiac arrest. However, rib fractures may be caused by caregivers using excessive force during chest compressions. Manual resuscitation may cause trauma to the chest-wall which can extend the recovery period, and increase the risk of acquiring pneumonia among surviving victims of cardiac arrest.

In addition, the depth and rate of chest compressions may vary due to fatigue of the caregivers after even a short period of time. This may cause inconsistency in the amount of blood flow that is sustained during resuscitation. Caregiver fatigue and inconsistent compressions may result in suboptimal coronary perfusion pressure, referring to the pressure gradient that drives coronary blood pressure. During cardiac arrest, maintaining a CPP of at least 15 mm Hg (millimeters of mercury pressure) is important for the return of spontaneous circulation (ROSC), the restoration of a pulse. Maintaining cerebral perfusion pressure, referring to the pressure gradient causing cerebral blood flow to the brain, is also important. In many instances, CPR must be performed for extended periods before additional medical assistance arrives to relieve a caregiver.

Accordingly, it may be desirable to provide CPR using a tool which will not become fatigued. An automated CPR device can be used to deliver chest-compressions during cardiac arrest without continued assistance from a rescuer. The automated CPR device may lower safety risks by allowing continued support in a caregiver's absence, and may allow a caregivers the flexibility to engage multiple victims. Automated CPR device features may include taking biometric and hemodynamic measurements and automatic adjustment of life support parameters. Biometric data may include information about the physical condition of a victim. Once attached to the chest-wall, the device can collect biometric data measurements of the victim, including bone density, body mass index (BMI), heart rhythms, and height and width or circumference of the chest cavity. In determining these parameters, the device may calculate chest compression parameters, such as compression timing, the depth of compressions necessary to avoid rib fractures and maximize perfusion during resuscitation efforts, other parameters, or any combination thereof.

An automated CPR device may be employed to determine biometric characteristics about a patient, and apply chest compressions with appropriate and consistent force and rhythm. Use of the device may be implemented by physicians, paramedics, nurses, and other critical care specialists in combat situations, humanitarian crises, or other situations. The automated CPR device can free caregivers to attend to other victims or to perform other procedures on the victim receiving the CPR. The automated CPR device may determine the pressure necessary to deliver adequate compressions and may deliver chest compressions accordingly. The automated device can operate within parameters that are specific to the victim. The device may take inputs from sensors, caregivers, or both, and calculate a compression force to achieve a safe coronary perfusion pressure or cerebral profusion pressure for the specific victim. For example, the automated CPR device may use data obtained about the victim to calculate a chest compression force and depth to reach a target coronary perfusion pressure of at least 15 mm Hg, or a target pressure range of 25 mm Hg+/−7 mm Hg, or another target value.

The automated CPR device can also minimize trauma caused by the use of excessive pressure during resuscitation efforts. The device can calculate an optimal pressure based on weight, bone density, the size of the chest cavity, or other parameters. The chest compressor can determine an amount of pressure required to deliver an adequate stroke volume while avoiding or minimizing rib fractures during chest compressions. The device can also minimize variations in the rate of chest compressions during CPR by performing uniform compressions. At the end of each compression cycle, the heart rhythm may be analyzed, for example using electrocardiogram (EKG or ECG) electrodes connected to a heart rate monitor. If a shockable heart rhythm that would benefit from defibrillation is detected, the device can sound an alarm prior to delivering a shock to the victim using a defibrillator integrated into the automated CPR device. The automated CPR device may include electrodes with adhesive pads, which may be used for administering shocks, detecting heart rhythms, or both. A defibrillator can also be attached to the device and operated separately from the automated CPR device.

This device may be compact, and may be designed for use in hostile environments or other emergency situations. The automated CPR device may be used during medical flight, military combat, at first aid stations, at hospitals, carried into disaster areas, or otherwise deployed. It can be stored in various facilities for emergency use. The CPR device may include a waterproof and damage proof case for application of the device in hostile environments. The device may include a transmitter to send a signal to a control center, in order to provide information on a victim's location and status. For example, a control center may include a hospital or urgent care center, which may prepare prior to arrival of the victim based on the biometric data transmitted by the automated CPR device.

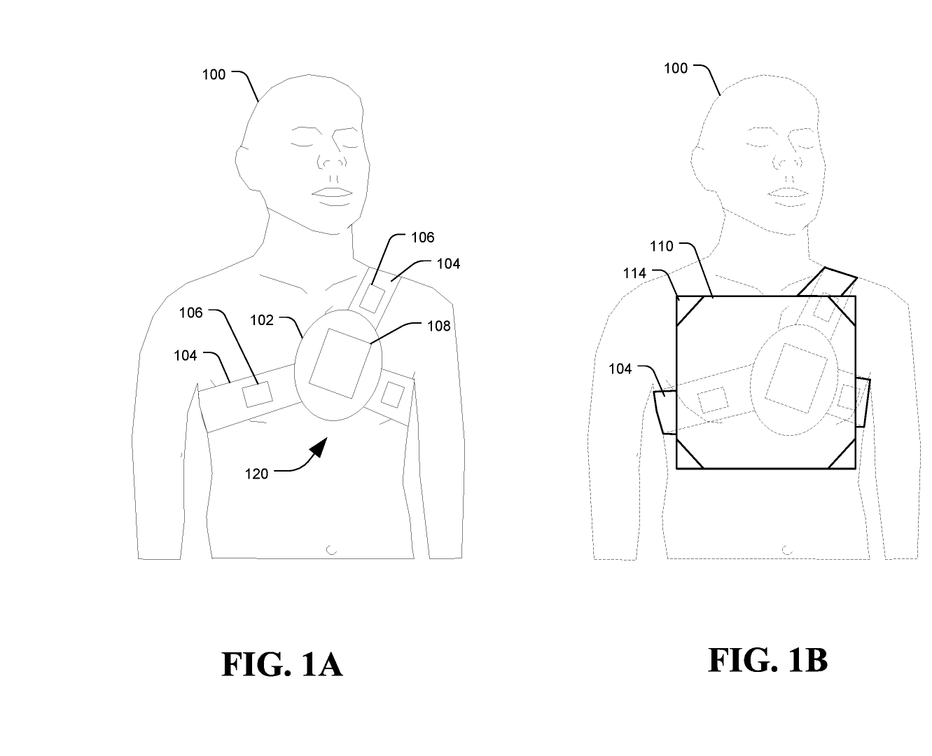

Referring to FIGS. 1A and 1B, an illustrative embodiment of an automated cardiopulmonary resuscitation (CPR) device is shown. FIG. 1A shows a view of the front of a victim 100 having an automated CPR device 102 on the victim's chest. The device may include a main housing 102, which may be strapped to a victim by means of one or more straps 104 which may be tightened around a victim's chest. FIG. 1B shows a view in which dashed lines are used to indicate foreground objects which are depicted as transparent. The device may also include a back panel 110, which may be placed under a victim's back. The straps 104 may attach to the back panel 110 to secure the device to the patient. For example, the device may include three straps 104, with one strap going around a victim's chest under the left arm, a second under the right arm, and a third strap leading over a victim's left shoulder. Additional or fewer straps may be used. The back panel 110 may include rubber or soft plastic coverings 114 on the corners or edges. The back panel 110 may be made of or coated in heavy duty water-proof plastic or other material.

A main housing 102 of the automated CPR device may include chest compression mechanism to perform chest compressions on a victim. Example chest compression mechanisms may include a pneumatic, hydraulic, or electrical system to drive a compressing pad into the victim's sternum, or a mechanism to contract a belt, such as belt 104, thereby compressing the victim's chest. For example, the compressing mechanism may include a gas-powered piston ending in a cushion, plunger, or other force dispersal device. The piston may deliver compressions to a victim's chest at a determined force and pressure. It may be desirable to position the main housing 102 or compression mechanism portion of the housing so that the compression pressure is concentrated on the left ventricle, the lower apex of the victim's heart. For example, the main housing 102 may be positioned approximately two finger spaces above the xiphoid process 120 of the victim's ribcage.

The housing 102 may also include a panel 108 for displaying information to a caregiver or receiving input from a user. For example, panel 108 may include a digital display for outputting vital statistics or biometric data of the victim, information on performance of the automated CPR device, or other information. The panel 108 may also include a button panel or touch-screen interface allowing a user to input information or, for example a height or weight of the victim, or to control the device, such as to pause compressions or set transmission information.

The housing 102 may further include processing components, such as one or more microprocessors, global positional system (GPS) sensors, wireless transmitters, medical sensors, sensor inputs, other components, or any combination thereof. For example, the housing 102 or straps 104 may include sensors 106 to detect biometric data from a victim. The biometric data can be used to modify device behavior based on the victim's specific condition. In some embodiments, sensors may monitor a victim's temperature, body fat, bone density, tissue perfusion and oxygenation, heart rhythm, or other information. Sensors may be in the form of one or more electrodes or sensors located on straps 104 of the device, in or on the housing 102, on a back panel 110, or otherwise communicatively coupled to the device. Sensors may include electric, magnetic, ultrasonic, or other measurement apparatuses.

Any appropriate sensors and methods may be used to collect the biometric data. For example, a peripheral densitometer for dual energy X-ray absorptiometry (DXA or DEXA) may be used to test a patient's bone density, either using a sensor attached to the housing 102 or straps 104, or by using a separate sensor to provide the information to the automated CPR device. For example, sensors could be used on portions of the body which may provide reliable bone density readings, such as at a heel or forearm, and transmit results to a receiver of the CPR device either wired or wirelessly. In an embodiment, a compact DXA/DEXA scanner connected to the housing 102 by a wire may be attached a victim's wrist, for example using a Velcro strap, to measure bone density. A Bioelectrical impedance analysis (BIA) sensor may be used to detect body fat or muscle density. Heart activity may be monitored with electrocardiography (ECG or EKG) via electrodes attached to the victim. Tissue oxygenation or tissue perfusion may be measured using a pulse oximeter, polarographic electrodes, spectrophotometry of hemoglobin using camera and image analysis systems, or other systems and methods. Blood pressure monitors, such as inflating arm cuffs, could be used to measure blood pressure or pulse. The depth or size of a victim's chest cavity may be determined based on an amount that straps 104 have been extended (e.g. to determine an approximate circumference of the chest), using a form of tomography to issue a penetrating wave to determine depth of the chest cavity, measuring magnetic fields (e.g. by including one or more magnets in the back panel 110 and measuring the magnetic field strength at the housing 102 to determine the chest depth), or using other methods. The size of the chest cavity may be used by the automated CPR device to determine an appropriate compression force. Computed tomography (CT or CAT scan) technology may also be used to obtain biometric data. Other sensors and embodiments are also possible.

The automated CPR device may use data obtained from sensors (e.g. for bone density, body fat, etc.) and information entered in via panel 108 (e.g. an approximated victim height or weight) to calculate a compression force to produce a safe coronary or cerebral perfusion pressure for the victim.

The straps may also include electrodes for delivering an electric shock to the victim, such as using a defibrillator. For example, one electrode may be included in a shoulder strap of the device, and another electrode may be included in a side strap. The straps 104, the sensors or electrodes 106, or both may be encased within an electrically-conducive material to obtain better electrical readings or for delivery of the defibrillating shock. For example, a solid or liquid gel may be used to coat the sensors or electrodes 106, or placed between the electrodes and the victim.

The housing 102 may also include a power source, such as a battery or electrical input, to provide power for the compressor, the processing components, the defibrillator, or any other component requiring power. In some embodiments, other power sources or fuels may also be used, such as gasoline, propane, other sources, or a combination thereof. For example, components such as the compressor may operate using a compressed gas canister to generate force and a battery to supply electrical power.

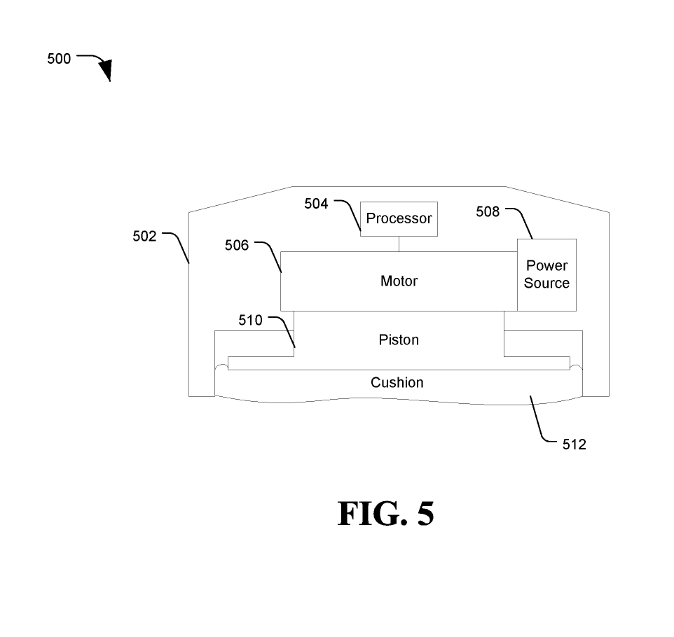

Turning to FIG. 5, an example of an automated CPR device 500 is shown, according to certain embodiments. In particular, FIG. 5 depicts a cross-sectional view of the housing portion 102 of the automated CPR device depicted in FIGS. 1A and 1B. CPR device 500 may include a housing 502 containing a processor 504, a motor 506, a power source 508, a piston 510, and a cushion or compression pad 512.

The CPR device may be positioned on a victim's chest, such as above the sternum, so that the cushion 512 is positioned against the chest. The cushion 512 may be of any soft material so as to distribute the force of chest compressions over the surface area of the cushion 512 and reduce the risk of injury to the victim. For example, the cushion 512 may include foam or cloth padding, a bladder containing a gel, soft plastic, rubber, or other materials. The cushion may be attached to the housing 502, such as to the sides of an inner wall of the housing.

The cushion may be pushed into a victim's chest during chest compressions by a piston 510. The piston may move up and down to produce the compressive force. In some embodiments other methods of chest compression than a motor-drive piston may be used. For example, the automated CPR device may tighten and loosen straps around the victim's chest in order produce the compressions.

The piston 510 may be powered by a motor 506. As discussed herein, the motor 506 may be a pneumatic, hydraulic, electrical, or any other type of motor which can be applied to drive the compressing pad 512 into the victim's sternum, or to contract a belt, thereby compressing the victim's chest. The motor 506 may be powered by a power source 508. Power source 508 may include a fuel source such as gasoline or natural gas. The power source 508 may include compressed gas. In some embodiments, the power source 508 may include a battery or power converter to power an electrical motor 506 or ignite a fuel source. In some embodiments, the power source 508 may be external to the housing 502, such as an external gas canister or an electrical plug that connects to the housing 502. An electrical power source 508 may also be used to perform defibrillation on a victim.

A processor 504 may control the motor 506 to regulate the chest compressions. The processor 504 may determine a compression force or depth based on data received from sensors or a control panel of the CPR device. Compressions may be performed at regular intervals, and the processor 504 may pause compressions periodically to allow a caregiver to perform artificial respiration, to detect heart rhythms, or for other purposes.

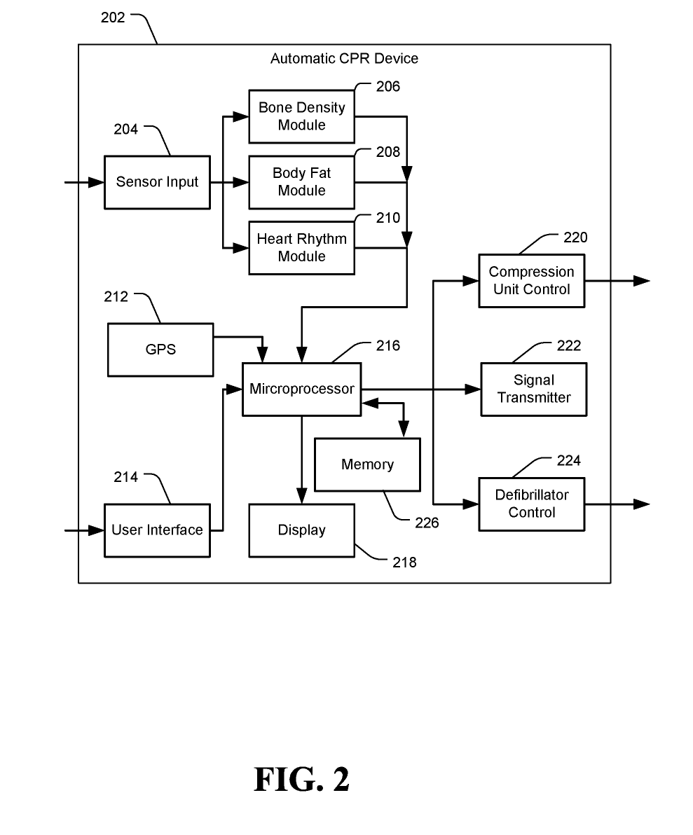

FIG. 2 depicts another illustrative embodiment of an automated cardiopulmonary resuscitation (CPR) device, generally designated 202. For example, the depicted automatic CPR device 202 may comprise a portion of the housing 102 of FIG. 1A containing computing elements, sensor inputs, and data outputs. In the depicted embodiment, the CPR device may include one or more sensor inputs 204, which may receive data from one or more sensors of the automatic CPR device. For example, the device may include sensors to detect biometric data, such as bone density, body fat, and heart rhythm of the victim, and may direct the sensor inputs to a bone density module 206, a body fat module 208, and a heart rhythm module 210. Other types of sensor inputs and data modules are also possible. Each module may perform calculations based on the sensor input, and pass the calculation results to a microprocessor 216 (such as processor 504 of FIG. 5). For example, each module may convert the sensor inputs into formats or numbers for use in calculations by the microprocessor (e.g. converting analog readings into numeric values). Modules may also perform some or all of the calculations for the values from the respective sensors. The microprocessor 216 may perform operations and calculations on the sensor inputs or data from the modules, such as using the sensor data to calculate compression force, determine when to apply defibrillator shocks, or send the data for display on a display unit 218. The microprocessor 216 may load firmware for operation of the automatic CPR device 202 from a memory 226, and may store sensor data or calculation outputs to the memory 226.

In some embodiments, measurements such as bone density and body composition of muscle and body fat may be used to determine an appropriate level of pressure to apply to a victim's chest through a compressor. For example if the victim is shown to have low bone density, a lower compression force may be applied. Similarly, if a victim has a high body fat reading, a higher force compression may be used. A heart rhythm reading may be used to determine whether to apply an electrical shock to a victim.

The CPR device 202 may also include a user interface 214, such as a keypad or touch screen, to receive input from a user. Information received at the user interface 214 may control device operations, such as instructing the CPR device to pause or resume operations, physical characteristics regarding the victim such as height or weight, or any other information. For example, a caregiver may input an estimated height and weight of a victim, and the CPR may use the information, possibly in combination from sensors such as a body fat sensor, to calculate a victim's body mass index (BMI). The BMI may be used to control a force of compressions on the victim.

The CPR device 202 may also include a global positioning system (GPS) sensor 212, which may determine a location of the automated CPR device and send positioning data to microprocessor 216. For example, the device 202 may determine the physical coordinates of the victim, allowing additional rescue personnel to locate the victim quickly.

The microprocessor 216 may send the positioning data through a signal transmitter 222. For example, the signal transmitter 222 may be a wireless data transmitter, a radio transmitter, satellite transmitter, or another form of wireless transmitter. The CPR device 202 may be configured to transmit the location data to a designated receiving address or location, or a caregiver may specify where to transmit data using the user interface 214. The signal transmitter may also be used to transmit biometric data regarding the victim. For example, the CPR device may transmit a victim's heart rate or temperature, so that a hospital or control center may be informed on a victim's condition.

As stated, the microprocessor 216 may perform calculations based on data received from any inputs, such as sensors input(s) 204 and user input(s) 214, and control operations of the CPR device 202. The microprocessor 216 may comprise one or more circuits configured to perform the operations described herein. The microprocessor 216 may be configured to executed methods according to instructions stored on a memory 226. For example, the microprocessor 216 may be connected to one or more volatile or non-volatile data storage mediums, which may store instructions, detected biometric data or other measurements, preprogrammed settings, calculated parameters, other data, or any combination thereof.

Based on detected measurements and stored instructions, the microprocessor 216 may control operation of the CPR device 202. As described, the microprocessor 216 may control the transmission of data via the signal transmitter 222. Further, the microprocessor 216 may control operation of a compression unit via a compression unit control interface 220. For example, the microprocessor 216 may control the force of compressions applied to a victim's chest based on detected or computed measurements about a victim, such as bone density, body fat, or body mass index. The microprocessor 216 may also control discharge of electrical energy using a defibrillator control interface 224. For example, the defibrillator control 224 may connect to electrodes on straps or otherwise located on or connected to the automated CPR device.

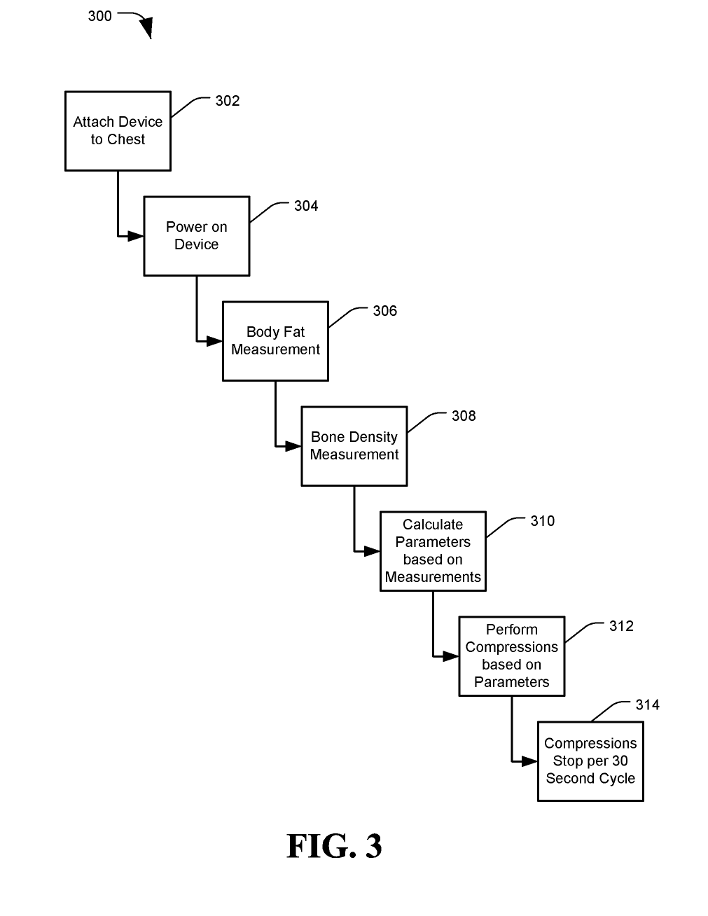

FIG. 3 depicts a flowchart of an illustrative method for an automated CPR device, generally designated 300. The method may include attaching the CPR device to a victim's chest, at 302. This may include reclining the victim into a supine position, removing or opening a victim's shirt, and sliding a back panel under the victim's back. The compression mechanism may be placed over the victim's chest, and one or more straps may be connected between the chest compression mechanism and the back panel to secure the compression mechanism onto the chest. Sensors may also be attached, such as placing a blood pressure cuff on the victim's arm or placing a pulse oximeter on the victim's finger. Depending on the location and type of sensors and electrodes contained in the CPR device, it may be important to have direct skin contact between the parts of the CPR device and the victim.

The method 300 may include powering on the automated CPR device, at 304. For example, this may include toggling a power switch for a device running on battery power, connecting another power source, establishing a flow of gas, or other steps. The automated CPR device may be instructed to begin CPR operations based on a manual input or automatically.

The method 300 may include performing measurements or taking readings from sensors, such as taking a body fat measurement, at 306. For example, sensors on the device may include an impedance meter which may be used to obtain a body fat reading, which may be transmitted to a processor of the CPR device. The method 300 may also involve performing a bone mineral density (BMD) measurement, at 308. For example, BMD may be measured using DXA scanners or ultrasound devices. Bone density data may also be transmitted to a processor of the CPR device. Other measurements may also be taken, such as temperature, heart rhythm, tissue perfusion, other measurements, or any combination thereof. Information may also be entered into the CPR device manually, such as an estimated height and weight of the victim.

The method 300 may include calculating parameters specific to the victim based on the obtained data, at 310. Data may include information obtained from sensors or information input by a caregiver or device operator. For example, the CPR device may calculate a chest compression force to use on the victim based on the readings. In an embodiment, the device may calculate a compression force or depth based on the measured body fat and bone density, the amount of force calculated to provide adequate compression depth for maintaining coronary or cranial profusion pressure without causing bone fractures or breakage. In another embodiment, the device may calculate a BMI for the victim based on an input height and weight of the victim, and measured body fat, and use the BMI and bone density to determine a compression force. For example, a processor may extract a chest compression force to employ from a chart having the victim's BMI rating on one axis and bone density rating on another axis. In some embodiments, the measured ratings may be run through an equation, with different measurements being given specific weight in the force calculation. Compression values may be looked up in a table stored to a memory of the device. Calculations to perform and compression values to employ may be determined based on software or firmware executed by one or more processors of the automated CPR device. Other embodiments are also possible.

The method 300 may include performing compressions based on the calculated parameters, at 312. The method 300 may also include pausing compressions on a timed cycle, such as a 30-second cycle, at 314. For example, a caregiver may take advantage of pauses during pauses in the compression cycle to manage the victim's airway. In some embodiments, the automated CPR device may monitor heart rhythm in between compression cycles to determine whether to administer defibrillation shocks to the victim.

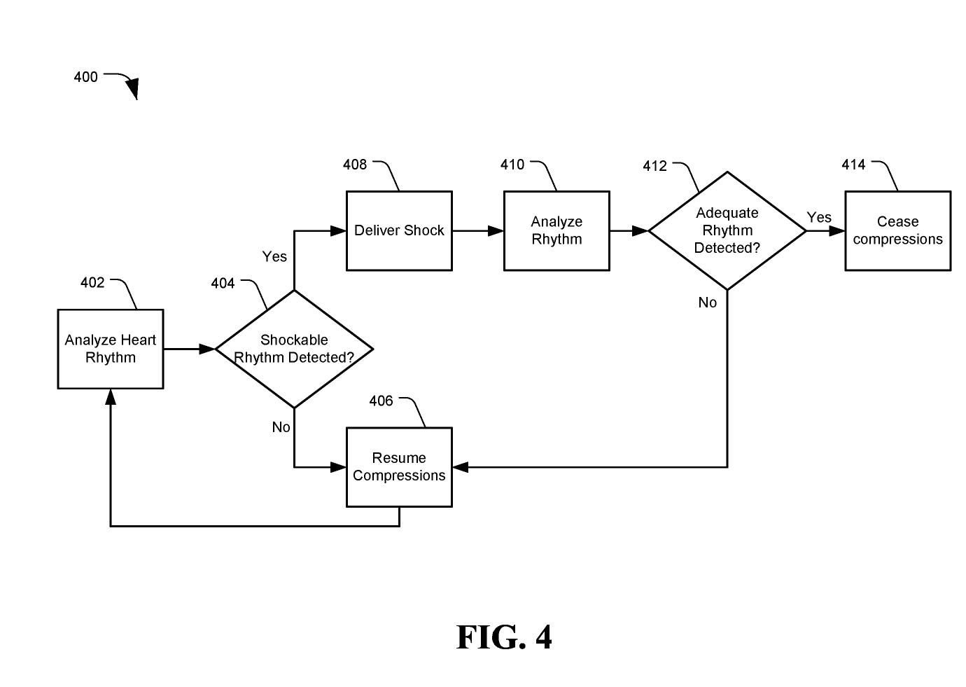

In many instances, CPR chest compressions are not sufficient to return a regular heart rhythm to a person suffering from cardiac arrhythmia, and defibrillating the victim may be required to restore a regular heart rhythm. FIG. 4 depicts a flowchart of another illustrative method for an automated CPR device, generally designated 400. The method 400 may include analyzing a victim's heart rhythm, at 402. For example, heart rhythm may be detected using electrodes located in the straps or elsewhere on the automated CPR device having skin contact with the victim. Heart rhythm may be monitored during a pause in chest compressions, for example.

The method 400 may include determining if a heart rhythm treatable with defibrillation (e.g. shockable cardiac arrhythmia) has been detected, at 404. If a shockable rhythm has not been detected, at 404, the method may include resuming chest compressions, at 406, and then analyzing the heart rhythm again at 402. Shockable rhythms may include detectable heart rhythms caused by an aberration in the electrical conduction systems of the heart, and may include ventricular tachycardia (v-tach), ventricular fibrillation (v-fib), and some cases of supraventricular tachycardia (SVT).

If a shockable rhythm is detected, at 404, the method 400 may include defibrillating the victim at 408, for example by delivering an electric current to the victim's heart through electrodes on the CPR device having contact with the victim. The automated CPR device may issue an audible or graphical warning (e.g. via a display panel) that caregivers should cease physical contact with the victim prior to delivering the electrical current. The method 400 may include analyzing the victim's heart rhythm again, at 410, and determining whether an adequate heart rhythm was detected (e.g. whether the cardiac dysrhythmia has been terminated and normal sinus rhythm reestablished), at 412.

If an adequate heart rhythm is not detected, at 412, the method 400 may include resuming chest compressions, at 406. If an adequate heart rhythm is detected, at 412, the method may involve ceasing chest compressions, at 414. For example, an adequate heart rhythm may mean that the victim has reestablished a heart rhythm within a normal rhythm range and chest compressions are no longer required. An automated CPR device may continue to monitor biometric data of the victim, or may stop monitoring.

In accordance with various embodiments, the methods and functions described herein may be implemented as one or more software or firmware programs running on a processor or controller device of the automated CPR device. Dedicated hardware implementations including, but not limited to, application specific integrated circuits, programmable logic arrays, and other hardware devices can likewise be constructed to implement the methods and functions described herein. Further, the methods described herein may be implemented as a computer readable storage medium or memory device including instructions that, when executed, cause a processor of the automated CPR device to perform the methods.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Moreover, although specific embodiments have been illustrated and described herein, it should be appreciated that any subsequent arrangement designed to achieve the same or similar purpose may be substituted for the specific embodiments shown.

This disclosure is intended to cover any and all subsequent adaptations or variations of various embodiments. Combinations of the above embodiments, and other embodiments not specifically described herein, will be apparent to those of skill in the art upon reviewing the description. Additionally, the illustrations are merely representational and may not be drawn to scale. Certain proportions within the illustrations may be exaggerated, while other proportions may be reduced. Accordingly, the disclosure and the figures are to be regarded as illustrative and not restrictive.

What is claimed is:

1. An apparatus comprising:
an automated chest compression device configured to be secured to a chest of an individual, the automated chest compression device including:
a compression cushion configured to be placed against the chest;
a motor to drive the compression cushion into the chest;
a processor configured to:
calculate one or more chest compression parameters based on data about the individual; and
control the motor to perform chest compressions based on the chest compression parameters.

2. The apparatus of claim 1 further comprising:
the automated chest compression device further including:
sensor electrodes configured to detect a heart rhythm of the individual;
a defibrillator;
the processor is further configured to:
monitor for a shockable heart rhythm based on data from the sensor electrodes; and
control the defibrillator to deliver an electric shock to the individual when a shockable heart rhythm is detected.

3. The apparatus of claim 2 comprising the processor further configured to:
control the motor to pause the chest compressions;
monitor for the shockable heart rhythm and control the defibrillator to deliver the electric shock during the pause in the chest compressions;
determine whether a normal heart rhythm is detected based on the data from the sensor electrodes; and
control the motor to resume the chest compressions when a normal heart rhythm is not detected.

4. The apparatus of claim 1 further comprising:
the automated chest compression device further including:
a global positioning system sensor to determine a location of the automated chest compression device; and
a signal transmitter; and
the processor further configured to transmit the location via the signal transmitter.

5. The apparatus of claim 4 comprising the processor further configured to transmit biometric data about the individual.

6. The apparatus of claim 1 further comprising:
the automated chest compression device further including:
a user interface configured to receive user inputs regarding physical characteristics of the individual; and
the processor further configured to calculate the one or more chest compression parameters based on the user inputs.

7. The apparatus of claim 1 further comprising:
the automated chest compression device further including:
one or more sensors configured to detect biometric data about the individual; and
the processor further configured to calculate the one or more chest compression parameters based on the biometric data.

8. The apparatus of claim 6 further comprising the one or more sensors include a body fat sensor.

9. The apparatus of claim 6 further comprising the one or more sensors include a bone density scanner.

10. The apparatus of claim 6 further comprising the one or more sensors are integrated into straps configured to secure the automated chest compression device to the chest of the individual.

11. The apparatus of claim 1 further comprising the one or more chest compression parameters include a chest compression depth.

12. The apparatus of claim 1 further comprising the one or more chest compression parameters include a chest compression pressure.

13. An apparatus comprising:
an automated cardiopulmonary resuscitation (CPR) device configured to be secured to a chest of an individual, the automated CPR device including:
a compression mechanism configured to perform chest compressions on the individual;
a defibrillator configured to deliver electrical shocks to the individual;
a processor configured to:
calculate one or more chest compression parameters based on data about the individual;
control the compression mechanism to perform chest compressions based on the chest compression parameters;
monitor the individual for a shockable heart rhythm; and
control the defibrillator to deliver an electric shock when a shockable heart rhythm is detected.

14. The apparatus of claim 13 further comprising:
the automated CPR device further including one or more sensors configured to detect biometric data from a group of sensors consisting of a heart rate sensor, a bone density scanner, a body fat sensor, a thermometer, a blood pressure monitor, and a tissue oxygenation sensor; and
the processor configured to calculate the one or more chest compression parameters based on the biometric data.

15. The apparatus of claim 14 further comprising:
the automated CPR device further including a signal transmitter; and
the processor further configured to transmit at least some of the biometric data obtained from the one or more sensors via the signal transmitter.

16. The apparatus of claim 15 further comprising:
the automated CPR device further including a global positioning system sensor to determine a location of the automated CPR device; and
the processor further configured to transmit the location via the signal transmitter.

17. The apparatus of claim 16 further comprising:
the automated CPR device further including a user interface configured to receive user inputs regarding physical characteristics of the individual; and
the processor further configured to calculate the one or more chest compression parameters based on the user inputs.

18. A method comprising:
attaching an automated cardiopulmonary resuscitation (CPR) device to a chest of an individual;
detecting biometric measurements from the individual via sensors of the automated CPR device;
calculating one or more chest compression parameters based on the biometric measurements via a processor of the automated CPR device; and
applying chest compressions to the individual via a chest compression mechanism of the automated CPR device based on the chest compression parameters.

19. The method of claim 18 further comprising:
detecting a heart rhythm of the individual via sensor electrodes of the automated CPR device;
determining whether a shockable heart rhythm is detected via the processor; and
administering an electrical shock to the individual via a defibrillator of the automated CPR device when the processor detects the shockable heart rhythm.

20. The method of claim 18 further comprising:
determining, via the processor, a location of the automated CPR device based on data from a global positioning system (GPS) sensor of the automated CPR device; and
transmitting the location via a signal transmitter of the automated CPR device.
